(19) United States
(12) Patent Application Publication
(10) Pub. No.: US 2013/0211496 A1
(43) Pub. Date: Aug. 15, 2013

(54) KNITTED STENT

(71) Applicant: Alfred Buck, Bondorf (DE)

(72) Inventor: Alfred Buck, Bondorf (DE)

(21) Appl. No.: 13/723,691

(22) Filed: Dec. 21, 2012

(30) Foreign Application Priority Data
Dec. 22, 2011 (DE) 10 2011 056 922.7

(51) Int. Cl. A61F 2/06; D04B 1/14; D04B 1/22

(52) U.S. Cl. CPC A61F 2/06; D04B 1/225; D04B 1/14
USPC 623/1.15; 66/170; 66/202

(57) ABSTRACT

The subject-matter of the invention relates to a knitted tube (100), in particular a stent (110) to be inserted into arterial blood vessels, comprising at least one circular knitted article (103) configured as knitted fabric as well as a method for producing a knitted tube (100), in particular a stent (110) to be inserted into arterial blood vessels, comprising the steps: production of a circular knitted article (103) configured as knitted fabric, wherein the produced circular knitted article (103) is reversed so that the originally internal side (106) of the circular knitted article (107) is arranged outside.

### FIELD OF THE INVENTION

The invention relates to a method for producing a knitted tube, in particular a stent to be inserted into arterial blood vessels, according to the preamble of claim 1.

### BACKGROUND OF THE INVENTION

The invention further relates to a knitted tube, in particular a stent to be inserted into arterial blood vessels, according to the preamble of claims 5 and 6.

The invention also relates to the application of a knitted tube according to the invention for medical purposes according to the preamble of claim 10.

Knitted tubes and methods for producing the same are known from the state of the art. Stents employing a knitted tube are also known from the state of the art.

DE 200 14 019 U1 discloses, for example, a stent to be inserted into arterial blood vessels, consisting of a tubular body which is permanently radially expandable, said tubular body consisting of a knitted article made of at least one thread with intermeshing loops.

### BRIEF SUMMARY OF THE INVENTION

It is an object of the present invention to provide a knitted tube, a method for producing the same and an application for the knitted tube which provide improved characteristics and fields of application compared to the state of the art.

These and other objects are achieved by a method according to claim 1, a knitted tube according to claims 5 and 6 and an application according to claim 10.

Advantageous developments of the invention are given in the dependent claims or below in connection with the description of the inventions.

The invention encloses the technical teaching that it is provided for a method for producing a knitted tube, in particular a stent to be inserted into arterial blood vessels, comprising the steps: producing a circular knitted article configured as knitted fabric, that the produced circular knitted article is reversed so that an originally internal side of the circular knitted article is arranged outside. The method according to the invention starts with the production of a technical knitted article that is configured, for example, as a sheet or a tube with several stitch courses connected to each other in a longitudinal direction so as to form a sheet or a tube extending in longitudinal direction. In one embodiment, recesses such as borings and the like are made in the tube. The tube or more generally the knitted article or the circular knitted article configured as knitted fabric is reversed after its production. The circular knitted article comprises one internal side or surface and one external side or surface after being produced. By reversing it, the originally internal surface becomes the external surface, and the originally external surface becomes the internal surface. For production reasons, the internal side of the circular knitted article is rougher than the external side during the production. This is due to the stitch formation and the fibers or wires crossing each other or lying on top of each other. By interlocking or interlooping the fiber or wire to form stitches, the internal side of the circular knitted article becomes rougher than the external side. The external side, however, is considerably smoother due to production reasons. To make the originally rougher internal side the external side, the circular knitted article is reversed. For this purpose, one end of the circular knitted article is passed through the circular knitted article. By passing one end through the whole length of the circular knitted article and beyond the other end, the position of the sides becomes reversed, so that the internal side becomes the external side and the external side becomes the internal side. The circular knitted article or knitted tube is quasi turned inside out or reversed.

In one embodiment, the circular knitted article is made of multiple fibers. The circular knitted article is preferably made of a single fiber by circular knitting. In one embodiment, multiple materials are integrated into one wire and/or one fiber. When choosing the thickness of a wire and/or fiber, it should be noted that the diameter of a wire and/or fiber is associated with its durability. To avoid the risk of breakage, the diameter of a wire and/or fiber should not be chosen too small. The wire and/or the fiber have a diameter from about 0.01 to 5 mm, preferably from about 0.05 mm to about 1 mm, and particularly preferably from about 0.1 mm to about 0.3 mm.

In one embodiment, it is therefore provided that a wire material, in particular a biocompatible metal material, is circular knitted for the production.

In another embodiment, it is provided that a memory material, in particular a biocompatible memory material, is circular knitted for the production.

Besides the choice of the wire or the fiber material, the properties of the knitted fabric are also influenced by the size and the density of the stitches, the density of the stitches being increasable in a shaping process step. This affects the properties of the knitted fabric, in particular the elasticity and thus also the resilience and the shock absorption. The knitted fabric has a stitch width from 0.01 mm to 50 mm, preferably from 0.5 mm to 20 mm, and particularly preferably from 3 mm to 8 mm. In particular, it has become clear that a weft-knitted fabric and in particular a circular knitted fabric is preferred. In this way, it is possible to realize a knitted article configured as a tube. In one embodiment, circular knitting is done in a first temperature range that differs from an application temperature range or second temperature range in which the circular knitted article is employed. The first temperature range preferably differs from the second temperature range by at least 2° C., preferably by at least 10° C., more preferably by at least 20° C., and most preferably by at least 25° C. The second temperature range is preferably in the range of the body temperature, that is, in the range around 37° C. plus/minus a tolerance range of about 5° C.

In particular it has been found advantageous that the stitches are knitted circular during the production and that the knitted fabric is present as a knitted tube. Recesses can be made in the tube. The density of the stitches can be increased if the knitted fabric is present as pressed knitted fabric. This is done in a preshaping process step. This has impacts on the property and in particular improves the elasticity and thus the resilience and damping properties. As a result of the knitted fabric, cavities are formed in one embodiment which, for example, can be used as spring deflections or into which other material can be introduced. The size of the cavities determines, for example, the properties with respect to rigidity, elasticity or bending elasticity, compressibility, resilience and damping. The elasticity and the damping as well as all the other parameters (e.g. dimensions) are chosen according to their application or intended use.

In still another embodiment of the present invention, it is provided that a stainless steel fiber is used as fiber, preferably a stainless steel fiber with an essentially circular cross-section having a diameter in the range from 0.01 mm to 5 mm or less, preferably from 0.05 mm or more to 4 mm or less, more preferably from 0.075 mm or more to 3.5 mm or less, and most preferably from 0.1 mm or more to 3 mm or less. The diameter can have any shape, a circular diameter shape has proven to be particularly advantageous.

In still another embodiment of the present invention, it is provided that a memory material fiber is used as fiber, preferably a memory material fiber with an essentially circular cross-section having a diameter in the range from 0.01 mm to 5 mm or less, preferably from 0.05 mm or more to 4 mm or less, more preferably from 0.075 mm or more to 3.5 mm or less, and most preferably from 0.1 mm or more to 3 mm or less. The diameter can have any shape, a circular diameter shape has proven to be particularly advantageous.

In one embodiment of the present invention, it is provided that a spring characteristic curve is determined by predetermining the stitch size and/or the fiber diameter. The spring characteristic curve is selected according to an application and can be linear or non-linear.

The circular knitted article or knitted tube preferably extends in a longitudinal direction. Accordingly, rows of interlooped stitches are formed in transverse directions. In one embodiment, for example, multiple tubes or circular knitted articles are connected to each other.

In another embodiment of the present invention, it is provided that a marking material, in particular a biocompatible marking material, is attached in and/or to the circular knitted article. In this way, a positioning of the produced knitted tube is easily determinable. Particularly in the case where the knitted tube is introduced into other hollow bodies or used as a stent, an implant or the like, the positioning is not easily discernible. A marking material is provided in order to mark the positioning. The marking material can be an optically distinguishable material or a material having properties which differ from those of a fiber material, for example with regard to shape, roughness, material properties and the like. In a preferred embodiment, a fiber is provided as marking material. The fiber is preferably attached to the circular knitted article. It is preferred to attach the fiber to an end of the circular knitted article. It is even more preferred to attach one fiber to each end of the circular knitted article so that a beginning and an end of the circular knitted article is discernible.

In a preferred embodiment, the circular knitted article is made of a single fiber. In one embodiment, the fiber is configured as a wire, for example as a steel wire or stainless steel wire. In another embodiment, the fiber is configured as a wire made of a memory material and/or titanium or a titanium alloy. The knitted tube is preferably used as a stent. When configured as a stent and/or an implant, the fiber is made of a biocompatible material. The biocompatible material is preferably not visible by or suited for nuclear spin tomography or other medical methods for imaging the interior of the human body. The marking material, however, is visible by appropriate screening, nuclear spin tomography, magnetic resonance imaging, MRT, CT, or the like. In one embodiment, it is in particular provided that the marking material has a ferromagnetic content or is completely configured as a ferromagnetic material. The material from which the rest of the stent is made is preferably composed of a non-ferromagnetic material. In another embodiment, the stent is in particular made of a titanium alloy. Preferably at least one material, preferably multiple or all materials are biocompatible or configured as a biomaterial. The materials for the stent are preferably configured as a biomaterial.

For the purposes of the present invention, a biomaterial or implant material is generally any synthetic or non-living material or substance that is used in therapeutic or diagnostic applications in medicine and that come into direct contact with biological body tissue. These materials can chemically, physically, and biologically interact with the corresponding biological systems. Broadly speaking, biomaterials include any materials that come in contact with the body during therapeutic or diagnostic methods, including temporary contacts via the external body surface, via body orifices and via mucous membranes accessible from the outside. They include in particular materials that are introduced into the interior of the body to remain there for a long time. The term biomaterial refers to the substantial properties of the material, in particular the chemical and physical properties. One characteristic of the biomaterial is a biocompatibility that results from its properties and comprises the functional similarity to endogenous structures as well as a an appropriate biological compatibility in the body.

The invention further includes the technical teaching that it is provided for a knitted tube, in particular a stent to be inserted into arterial blood vessels, comprising at least one circular knitted article configured as knitted fabric, that the circular knitted article is produced in accordance with a method according to the invention.

The invention also includes the technical teaching that it is provided for a knitted tube, in particular a stent to be inserted into arterial blood vessels, comprising at least one circular knitted article configured as knitted fabric, that the circular knitted article has an internal side and an external side, the external side being rougher than the internal side.

In this way it is possible to produce a knitted tube that is particularly suitable for high temperatures, aggressive environments and high stress. In particular, the knitted tube is designed for an application in a temperature range that is preferably between −80° C. or higher and 700° C. or lower, more preferably between −20° C. or higher and 100° C. or lower, and most preferably between 30° C. or higher and 40° C. or lower, in particular for body temperature ranges.

In one embodiment of the present invention, it is provided that the circular knitted article has an internal side and an external side, the external side being rougher than the internal side. In circular knitting, the internal side is rougher than the external side. By reversing the knitted tube or turning it inside out, the rougher side is turned to the outside. The rougher side is thus outside. Roughening is not required. The roughness depends on the interlacing loops of the circular knitted article. Due to the interlacing loops on the outside, the external side is rougher. The external side is particularly advantageous for the application as a stent because it allows an improved joining to the vein walls or arterial blood vessel walls by healing.

Still another embodiment of the present invention provides that the circular knitted article is made of at least one fiber and/or one thread consisting of a metal material, in particular a biocompatible metal material. It is preferred that the circular knitted article is made of a single thread or a single fiber. The fiber or the thread is in particular made of a metal material. It is further preferred that the circular knitted article is made of a single fiber consisting of a biocompatible material.

Still another embodiment of the present invention provides that the circular knitted article is made of at least one fiber and/or one thread consisting of a memory material, in particular a biocompatible memory material.

It is preferred that the circular knitted article is made of a single thread or a single fiber. The fiber or the thread is in particular made of a memory material. It is further preferred that the circular knitted article is made of a single fiber consisting of a biocompatible memory material.

Still another embodiment of the present invention provides that the circular knitted article comprises at least one marking material, in particular a biocompatible marking material. In particular, the marking material is configured as a thread or a fiber. The fiber is preferably arranged transverse to the extension direction of the knitted tube and preferably extends in circumferential direction on the knitted tube. The marking material is for example configured as a ring, a helix or in a ring-shaped or helical manner.

The invention further includes the technical teaching that a knitted tube according to the invention is intended for use for medical purposes, in particular for use as an implant, a stent or the like. The circular knitted article is preferably made of a fiber consisting of a biocompatible material, preferably of a biocompatible memory material. Supporting elements in the interior of the knitted tube are not provided, so that the knitted tube is configured without any supporting elements. The knitted tube is thus configured self-supporting. During insertion, the knitted tube is radially compressed. Then the knitted tube is positioned. Once in its position, the knitted tube unfolds to the desired size, that is, it radially extends again. In one embodiment, this is caused by the memory effect of the used memory material. In another embodiment, this is due to the elastic property of the knitted tube. The knitted tube is reversibly deformable in a radial direction. In one embodiment, the knitted tube is elastically reversibly deformable. In another embodiment, the knitted tube is reversibly deformable by the memory effect. In this case, the knitted tube does not become permanently deformed. The knitted tube is compressed before inserting it into a blood vessel. This is done, for example, by drawing the knitted tube into an outer shell, for example a guiding catheter. Then, the knitted tube is positioned in the blood vessel. After removal of the balloon catheter, the knitted tube expands to the desired size. At least one outer end of the knitted tube is preferably domed, for example by appropriate arrangement of the stitches whose loop portion points to the end. Another embodiment provides a compressible material that forms an edge of the knitted tube. The compressible edge material is, in one embodiment, integrated into the marking material, in particular formed in one piece with the latter. Another embodiment provides that the stent comprises multiple knitted tubes connected to each other. In one embodiment, the knitted tubes are configured differently. In one embodiment, it is provided that the stent comprises a knitted tube with a rougher external side to which is attached a knitted tube with a smoother external side. In a preferred embodiment, at least two knitted tubes with rougher external sides are provided, preferably on the end regions of the complete stent. At least one knitted tube with a smoother external side is arranged between them. The knitted tubes are preferably welded together. One edge of the knitted tube is preferably welded in such a manner that no fiber sticks out. In the edge region of the knitted tube, one fiber is similarly welded to the base body so that no sharp projections extend from the base body or the stent.

Further methods to improve the invention are specified in the subclaims or become apparent from the following description of at least one embodiment of the invention that is schematically represented in the figures. All and any of the features and/or advantages disclosed in the claims, the description or the drawings, including structural details, spatial arrangements and process steps, can be essential to the invention both independently and in a great variety of combinations.

### DETAILED DESCRIPTION OF ILLUSTRATIVE EMBODIMENTS

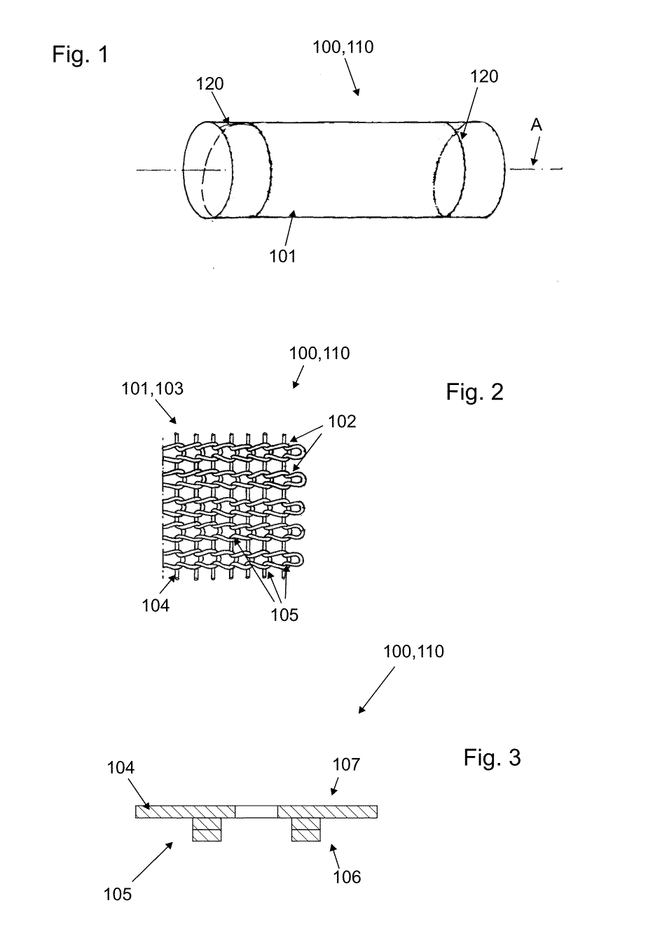

FIG. 1 shows schematically a knitted tube configured as a stent;

FIG. 2 shows schematically a top view of the stent according to FIG. 1; and

FIG. 3 shows schematically and in a very simplified manner a cross-sectional view of the internal and external side of the knitted tube according to FIGS. 1 and 2.

FIG. 1 shows schematically a knitted tube 100 configured as a stent 110. In an unloaded state, the knitted tube comprises an essentially cylindrical base body 101 with an axis of symmetry A. The base body 101 is made of a single wire as a circular knitted article with stitches 102 (see FIG. 2). The wire is configured as a metal wire from a memory alloy or a memory metal such as nitinol, biometal or the like. The wire changes its configuration in a particular temperature range. At a lower temperature, the stent 110 has therefore a smaller diameter, and at a transition temperature, it expands radially to a larger diameter. The stitches are produced by circular knitting. Furthermore, the knitted tube 110 comprises a marking material 120 on each end which is configured as a helical fiber. The fibers are incorporated into the base body 101. The marking material 120 is made of an other material than the base body 101. The marking material 120 only serves to mark the position of the stent 110 so that it becomes clearly distinguishable from the base body 101.

FIG. 2 shows schematically a top view of the stent 110 according to FIG. 1. The stent 110 comprises the tubular or ring-shaped elongated base body 101 that is represented in FIG. 2 in a side view. The base body 101 consists of a knitted article, more precisely a circular knitted article 103 that, in the present embodiment, only consists of a single fiber 104. This fiber 104 is knitted with needles so that it forms multiple circumferential turns each comprising a multitude of stitches 102. The fiber 104 consists of a wire made of a memory material, more precisely a memory metal material which is coated, where required. The wire size is between about 0.1 mm and 0.2 mm. The fiber forms interlacings 105 at any points where stitches 102 are produced. These interlacings 105 produce roughness on one side, shell or surface of the stent 110. The interlacings 105 are arranged in such a manner that there is one smoother side 107 and one rougher side 106, as is shown in a very simplified manner in FIG. 3.

FIG. 3 shows schematically and in a very simplified manner a cross-sectional view of an internal and an external side of the knitted tube 100 according to FIGS. 1 and 2. An interlacing 105 is here shown in a detailed and very schematic manner. The fiber 104 crosses itself in the region of the interlacing 105 so that it produces a higher roughness in the one direction, that is, on the one side, than on the other side. In the present embodiment, the internal side 106 is rougher because the fiber 104 superposes in that direction. The external side 107 is smoother. In circular knitting, the internal side of the knitted tube 100 is the rougher side. The rougher internal side 106 becomes the external side 107 by reversing the knitted tube 100 or turning it inside out. When the knitted tube 100 is used as a stent 110, it is advantageous if the external side 107 is rougher because it supports the joining to the blood vessel walls by healing. It is also advantageous that the internal side 106 facing to the flow is smoother or less rough to ensure that the flow is influenced as little as possible.

### LIST OF REFERENCE SIGNS

100 knitted tube

101 base body

102 stitches

103 (circular) knitted article

104 fiber

105 interlacing

106 (internal) side

107 (outer) side

110 stent

120 marking material

A axis of symmetry (longitudinal direction)

What is claimed is:

1. A method for the production of a tubular knitting, in particular a stent to be inserted into arterial blood vessels, comprising the following steps:
producing a circular knitted article configured as knitted fabric, wherein the produced circular knitted article is reversed so that the originally internal side of the circular knitted article is arranged outside.

2. The method according to claim 1, wherein
a wire material, is circular knitted for the production.

3. The method according to claim 1, wherein
a memory material is circular knitted for the production.

4. The method according to claim 1, wherein
a marking material is attached in and/or to the circular knitted article.

5. A knitted tube to be inserted into arterial blood vessels, comprising at least one circular knitted article configured as knitted fabric, wherein
the circular knitted article is produced in accordance with the method of claim 1.

6. A knitted tube to be inserted into arterial blood vessels, comprising at least one circular knitted article configured as knitted fabric, wherein
the circular knitted article has an internal side and an external side, wherein the external side is rougher than the internal side.

7. The knitted tube according to claim 5, wherein
the circular knitted article is produced of at least one fiber consisting of a metal material.

8. The knitted tube according to claim 5, wherein
the circular knitted article is produced of at least one fiber consisting of a memory material.

9. The knitted tube according to claim 5, wherein
the circular knitted article comprises at least one marking material.

10. Application of a knitted tube according to claim 1 for medical purposes, in particular for application as an implant, and/or a stent.

11. Method according to claim 2, wherein the wire material is a biocompatible metal material.

12. Method according to claim 3, wherein the memory material is a biocompatible memory material.

13. The method according to claim 4, wherein the marking material is a biocompatible marking material.

14. The knitted tube of claim 5, wherein the knitted tube is a stent.

15. The knitted tube of claim 6, wherein the knitted tube is a stent.

16. The knitted tube of claim 7, wherein the metal material is a biocompatible metal material.

17. The knitted tube of claim 8, wherein the memory material is a biocompatible memory material.

18. The knitted tube of claim 9, wherein the marking material is a biocompatible marking material.
